(19) United States
(12) Patent Application Publication
(10) Pub. No.: US 2009/0062686 A1
(43) Pub. Date: Mar. 5, 2009

(54) Physiological condition measuring device

(76) Inventors: Roderick A. Hyde, Redmond, WA (US); Muriel Y. Ishikawa, Livermore, CA (US); Jordin T. Kare, Seattle, WA (US); Eric C. Leuthardt, St. Louis, MO (US); Royce A. Levien, Lexington, MA (US); Lowell L. Wood, JR., Bellevue, WA (US); Victoria Y.H. Wood, Livermore, CA (US); Dennis J. Rivet, Portsmouth, VA (US)

Correspondence Address: IV - SUITER SWANTZ PC LLO, 14301 FNB PARKWAY , SUITE 220, OMAHA, NE, 68154, US

(21) Appl. No.: 11/906,122

(22) Filed: Sep. 28, 2007

Related U.S. Application Data
(63) Continuation-in-part of application No. 11/899,606, filed on Sep. 5, 2007.

(51) Int. Cl. A61B 5/00; A61B 5/107; G06K 9/36; G01N 1/22; G08B 23/00

(52) U.S. Cl. 600/558; 600/300; 600/559; 600/587; 705/3; 382/118; 382/117; 422/84; 340/573.7

(57) ABSTRACT

A method may include providing an output comprising a presentation format to an end user. The output may be provided for user-based interaction. An interactive response from the end user may be measured in response to the presentation format of the output, where the interactive response may be indicative of at least one physiological condition regarding the end user.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)).

### RELATED APPLICATIONS

For purposes of the USPTO extra-statutory requirements, the present application constitutes a continuation-in-part of U.S. patent application Ser. No. 11/899,606, entitled PHYSIOLOGICAL CONDITION MEASURING DEVICE, naming Roderick A. Hyde; Muriel Y. Ishikawa; Jordin T. Kare; Eric C. Leuthard; Royce A. Levien; Lowell L. Wood Jr.; and Victoria Y. H. Wood as inventors, filed 5 Sep. 2007, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin Benefit of Prior-Filed Application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### BACKGROUND

Portable electronic devices have become ubiquitous in modern society. Because of the rapid and increasing miniaturization of components, such devices have become increasingly sophisticated. It would be advantageous to leverage these devices to make valuable determinations about the health and well being of a user. For example, in many cases an annual doctor's visit should be supplemented with more frequent monitoring throughout the year, especially when the sophistication of modern medicine allows for increasingly effective treatments with early diagnosis and analysis. Further, many people with existing conditions would benefit from periodic monitoring of physiological characteristics that may have an impact on their health. Other users may desire information regarding their progress toward a goal state, such as weight loss, or the like.

### SUMMARY

A method includes providing an output including but not limited to a presentation format to an end user. The output may be provided for user-based interaction. An interactive response from the end user may be measured in response to the presentation format of the output, where the interactive response may be indicative of at least one physiological condition regarding the end user. In addition to the foregoing, other method aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

A system includes a means for providing an output including but not limited to a presentation format to an end user. The output may be provided for user-based interaction. The system may further include a means for measuring an interactive response from the end user in response to the presentation format of the output. The interactive response may be indicative of at least one physiological condition regarding the end user. In addition to the foregoing, other system aspects are described in the claims, drawings, and text forming a part of the present disclosure.

A system includes circuitry for providing an output including but not limited to a presentation format to an end user. The output may be provided for user-based interaction. The system may further include circuitry for measuring an interactive response from the end user in response to the presentation format of the output. The interactive response may be indicative of at least one physiological condition regarding the end user. In addition to the foregoing, other system aspects are described in the claims, drawings, and text forming a part of the present disclosure.

The foregoing is a summary and thus may contain simplifications, generalizations, inclusions and/or omissions of detail; consequently, those skilled in the art will appreciate that the summary is illustrative only and is NOT intended to be in any way limiting. Other aspects, features, and advantages of the devices and/or processes and/or other subject matter described herein will become apparent in the teachings set forth herein.

### BRIEF DESCRIPTION OF THE FIGURES

FIG. 1 is a schematic of a communication device including a processing unit and an image capture device.

FIG. 2 illustrates an operational flow representing example operations related to measuring at least one physiological condition for an end user.

FIG. 3 illustrates an alternative embodiment of the operational flow of FIG. 2.

FIG. 4 illustrates an alternative embodiment of the operational flow of FIG. 2.

FIG. 5 illustrates an alternative embodiment of the operational flow of FIG. 2.

FIG. 6 illustrates an alternative embodiment of the operational flow of FIG. 2.

FIG. 7 illustrates an alternative embodiment of the operational flow of FIG. 2.

FIG. 8 illustrates an alternative embodiment of the operational flow of FIG. 2.

FIG. 9 illustrates an operational flow representing example operations related to measuring at least one physiological condition for an end user.

FIG. 10 illustrates an operational flow representing example operations related to measuring at least one physiological condition for an end user.

FIG. 11 illustrates an alternative embodiment of the operational flow of FIG. 2.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

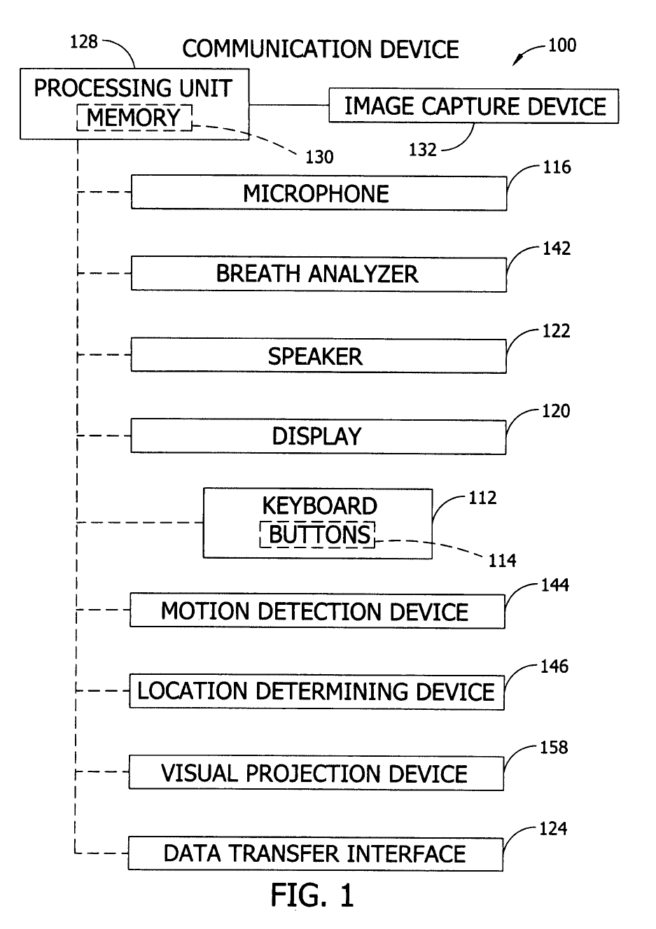

Referring now to FIG. 1, a device 100 is illustrated. The device 100 may comprise a cellular telephone, a personal digital assistant (PDA), a portable game player, a portable audio player, or another type of device, such as an iPod marketed by Apple Inc. in Cupertino, Calif. The device 100 generally represents instrumentality for user-based interaction. User-based interaction may be accomplished electronically, e.g., with an electronic circuit and/or another set of electrical connections for receiving an input (such as a user-generated command) and providing an output (such as an audio, video, or tactile response). An electronic circuit may comprise an Integrated Circuit (IC), such as a collection of interconnected electrical components and connectors supported on a substrate. One or more IC's may be included with the device 100 for accomplishing a function thereof.

The device 100 may comprise a printed circuit board having conductive paths superimposed (printed) on one or more sides of a board made from an insulating material. The printed circuit board may contain internal signal layers, power and ground planes, and other circuitry as needed. A variety of components may be connected to the printed circuit board, including chips, sockets, and the like. It will be appreciated that these components may be connected to various types and layers of circuitry included with the printed circuit board.

The device 100 may include a housing, such as a protective cover for at least partially containing and/or supporting a printed circuit board and other components that may be included with the device 100. The housing may be formed from a material such as a plastic material comprising a synthetic or semi-synthetic polymerization product. Alternatively, the housing may be formed from other materials, including rubber materials, materials with rubber-like properties, and metal. The housing may be designed for impact resistance and durability. Further, the housing may be designed for being ergonomically gripped by the hand of a user.

The device 100 may be powered via one or more batteries for storing energy and making it available in an electrical form. Alternatively, the device 100 may be powered via electrical energy supplied by a central utility (e.g., via AC mains). The device 100 may include a port for connecting the device to an electrical outlet via a cord and powering the device 100 and/or for charging the battery. Alternatively, the device 100 may be wirelessly powered and/or charged by placing the device in proximity to a charging station designed for wireless power distribution.

User-based interaction may be accomplished utilizing a variety of techniques. The device 100 may comprise a keyboard 112 including a number of buttons. The user may interact with the device by pressing a button 114 to operate an electrical switch, thereby establishing an electrical connection in the device 100. The user may issue an audible command or a command sequence to a microphone 116. The device 100 may comprise an electrode for measuring activity of a user's nervous system and/or for providing stimulation to the nervous system. The electrode may comprise an electrically conductive element placed in contact with body tissue for detecting electrical activity and/or for delivering electrical energy.

User-based interaction may be facilitated by providing tactile feedback to the user. The device 100 may include various electrical and/or mechanical components for providing haptic feedback, such as the feeling of a button press on a touch screen, variable resistance when manipulating an input device (e.g., a joystick/control pad), and the like. The device 100 may provide feedback by presenting data to the user in visual form via a display 120, in audible form via a speaker 122, and with other audio/visual playback mechanisms as desired.

The display 120 may comprise a Liquid Crystal Display (LCD), a Light Emitting Diode (LED) display, an Organic Light Emitting Diode (OLED) display, a Cathode Ray Tube (CRT) display, a fiber optic display, and other display types. It will be appreciated that a variety of displays may be utilized to present visual information to a user as desired. Similarly, a variety of mechanisms may be utilized to present audio information to the user of the device 100. The speaker 122 may comprise a transducer for converting electrical energy (e.g., signals from an electronic circuit) into mechanical energy at frequencies around the audible range of a user.

The device 100 may comprise a communication device configured for communication transfer. The communication device may be utilized to facilitate an interconnection between the user and one or more other parties. The communication device may provide for the transmission of speech data between the user and another individual by converting speech to an electric signal for transmission from one party to another. The communication device may provide for the transmission of electronic data between the device 100 and another device by transmitting data in the form of an electric signal from one device to another. The communication device may connect with another party and/or another device via a physical connection and/or via a wireless connection.

The communication device may connect with another party or another device via a physical interconnection outlet, e.g., a telephone jack, an Ethernet jack, or the like. Alternatively, the communication device may connect with another party and/or another device via a wireless connection scheme, e.g., utilizing a wireless network protocol, radio transmission, infrared transmission, and the like. The device 100 may include a data transfer interface 124 for connecting to one or more parties utilizing either a physical connection or a wireless connection. The data transfer interface 124 may comprise a physical access point, such as an Ethernet port, a software-defined transmission scheme, such as executable software for formatting and decoding data transmitted and received, as well as other interfaces for communication transfer as desired.

The device 100 may include an antenna for radiating and/or receiving data in the form of radio energy. The antenna may be fully or partially enclosed by the housing, or external to the housing. The device 100 may utilize the antenna to transmit and receive wirelessly over a single frequency in the case of a half-duplex wireless transmission scheme, or over more than one frequency in the case of a full-duplex wireless transmission scheme. The antenna may be constructed for efficiently receiving and broadcasting information over one or more desired radio frequency bands. Alternatively, the device 100 may include software and/or hardware for tuning the transmission and reception of the antenna to one or more frequency bands as needed.

The device 100 may broadcast and/or receive data in an analog format. Alternatively, the device 100 may broadcast and/or receive data in a digital format. The device 100 may include analog-to-digital and/or digital-to-analog conversion hardware for translating signals from one format to another. Additionally, the device 100 may include a Digital Signal Processor (DSP) for performing signal manipulation calculations at high speeds. A processing unit 128 may be included with the device 100 and at least substantially enclosed by the housing. The processing unit 128 may be electrically coupled with the microphone 116, the speaker 122, the display 120, the keyboard 112, and other components of the device 100, such as the data transfer interface 124. The processing unit may comprise a microprocessor for receiving data from the keyboard 112 and/or the microphone 116, sending data to the display 120 and/or the speaker 122, controlling data signaling, and coordinating other functions on a printed circuit board.

The processing unit 128 may be capable of transferring data relating to the status of a user (e.g., a measurement of a physiological condition). The device 100 may be connected to a variety of transmitting and receiving devices operating across a wide range of frequencies. The device 100 may be variously connected to a number of wireless network base stations. Alternatively, the device 100 may be variously connected to a number of cellular base stations. In this manner, the device 100 may be able to establish and maintain communication transfer between the user and one or more other parties while the device 100 is geographically mobile. The processing unit 128 may command and control signaling with a base station. The communication device may transmit and receive information utilizing a variety of technologies, including Frequency Division Multiple Access (FDMA), Time Division Multiple Access (TDMA), and Code Division Multiple Access (CDMA). The communication device may comprise a variety of telephony capable devices, including a mobile telephone, cellular telephone, a pager, a telephony equipped hand-held computer, personal digital assistant (PDA), and other devices equipped for communication transfer.

The device 100 may include a variety of components for information storage and retrieval, including Random Access Memory (RAM), Read Only Memory (ROM), Electrically Erasable Programmable Read Only Memory (EEPROM), and programmable nonvolatile memory (flash memory). The processing unit 128 may be utilized for controlling data storage and retrieval in the memory of the device 100. The processing unit 128 may also be utilized for formatting data for transmission between the device 100 and one or more additional parties. The processing unit 128 may comprise memory 130, such as the storage and retrieval components described. The memory 130 may be provided in the form of a data cache. The memory 130 may be utilized to store data relating to the status of a user (e.g., a measurement of a physiological condition). The memory 130 may be utilized for storing instructions executable by the processing unit 128. Such instructions may comprise a computer program native to the device 100, software acquired from a third party via the data transfer interface 124, as well as other instructions as desired.

The device 100 may comprise an image capture device 132, such as a camera for capturing a single image (e.g., a still image) or a sequence of images (e.g., a movie). The image capture device 132 may be electrically coupled to the processing unit 128 for receiving images. An image captured by the camera may be stored by the information storage and retrieval components of the device 100 as directed by the processing unit 128. An image may be converted into an electric signal and transmitted from one party to another via an interconnection between the user and one or more other parties (e.g., via a physical or wireless connection).

The device 100 may be equipped for measuring a physiological condition. The measurements may be performed in the background without explicit user commands. Further, the measurements may be performed in a passive manner (e.g., without user instructions and/or without a user's knowledge) or in an active manner (e.g., according to user instructions and/or with a user's knowledge). A physiological measurement may be utilized for making a determination about the status of a user (e.g., a user's health and/or wellbeing). Alternatively, a physiological measurement may be utilized for directing functioning of the device 100. For instance, in the case of the cellular telephone, the act of raising the volume of a user's voice may trigger a response from the telephone. The response may comprise raising the volume of audio provided by the speaker 122. It will be appreciated that physiological measurements taken by the device 100 in either an active manner or a passive manner may be utilized for a variety of purposes.

An image capture device 132, such as a camera may be utilized to capture an image of the user. The camera may then provide the image to the processing unit 128, which may analyze the image. The processing unit 128 may analyze the image utilizing a variety of optical measurement techniques. For example, optical measurements may be taken of various facial features for facial recognition. Alternatively, the camera may be utilized to capture an image of a user's eye. The processing unit 128 may analyze the image and perform a retinal scan of the of the user's eye.

The recognition of facial features and the retinal scan may be utilized for a variety of purposes, including identification of the user and/or monitoring of the user's status (e.g., the user's overall health and/or wellbeing). For instance, images may be examined for various shapes and sizes (e.g., mole and/or birthmark dimensions), tones and hues (e.g., skin color/pallor), and other characteristics indicative of a user's status. It will be appreciated that the forgoing list is exemplary and explanatory only, and images captured by the image capture device 132 may be analyzed to identify any physiological state or condition having visually identifiable features.

The electrode may be coupled with the processing unit 128 for performing a transdermal measurement through or by way of the skin. Alternatively, another type of device may be utilized for performing such a measurement. These transdermal measurements may be utilized for determining the amount of perspiration of a user, determining the health of a user's nervous system, and for other purposes as needed. Further, it will be appreciated that other equipment may be utilized for taking a measurement through the user's skin. A needle may be utilized to probe a user for a blood sample to determine a blood sugar level. Alternatively, a probe may be utilized to test the sensitivity of the user to a touch stimulus.

The microphone 116 may be utilized for measuring a user's vocal output and/or the surroundings of the user to determine the user's status. For example, the user's voice may be analyzed for voice recognition (i.e., to determine the identity of the user). Alternatively, the microphone 116 may be utilized for providing the processing unit 128 with audio data from a user to measure a physiological condition. For instance, the microphone 116 may be utilized for measuring a user's vocal output to determine the mood of the user. A warning may be issued to the user if the user's overall mood is determined to be contrary to a known or predicted health condition. For example, a user suffering from high blood pressure may be warned of undue exertion if a vocal stress determination is found to be at a dangerous level. In another instance, the microphone 116 may be utilized for measuring a user's audio output to determine a user's level of respiration (e.g., a breathing rate).

Alternatively, the microphone 116 may be utilized to collect information about a user's surroundings in an effort to identify the user's environment and/or characteristics thereof. The device 100 may report such characteristics to the user, or to another party as desired. It will be appreciated that the microphone 116 may be utilized to collect a variety of physiological and environmental data regarding a user. Further, it will be appreciated that the processing unit 128 may analyze this data in a number of different ways, depending upon a desired set of information and/or characteristics.

The device 100 may be equipped with a breath analyzer 142 (e.g., a microfluid chip) electrically coupled to the processing unit 128. The breath analyzer 142 may be utilized for receiving and analyzing the breath of a user. For example, the breath analyzer 142 may be utilized for sampling a user's breath to determine/measure the presence of alcohol on the user's breath. The processing unit 128 may then analyze measurements taken by the breath analyzer 142 to determine a blood-alcohol level for the user. The device 100 may be utilized to report on a level of alcohol as specified for a particular user (e.g., an unsafe and/or illegal level). Further, the breath analyzer 142 may be utilized for other purposes as well, including detecting the presence of chemicals, viruses, and/or bacteria on a user's breath. Other characteristics of the user's breath may be monitored and reported on as well, including temperature, moisture content, and other characteristics as needed.

The device 100 may be equipped with a motion detection device 144 electrically coupled to the processing unit 128. The motion detection device 144 may comprise an accelerometer, or another device for detecting and measuring acceleration, vibration, and/or other movements of the device 100. When the device 100 is held or retained by the user, movements of the user may be measured by the accelerometer and monitored by the processing unit 128. The processing unit 128 may be utilized to detect abnormal movements, e.g., tremors that may be indicative of Parkinson's disease, and the like. The processing unit 128 may also be utilized to detect information regarding a user's motion, including gait, and stride frequency (e.g., in the manner of a pedometer).

Alternatively, the processing unit 128 may be utilized to detect abnormal movements comprising sudden acceleration and/or deceleration indicative of a movement that may be injurious to the user. For example, violent deceleration could be indicative of a car accident, while sudden acceleration followed by an abrupt stop could be indicative of a fall. It will be appreciated that the aforementioned scenarios are exemplary and explanatory only, and that the motion detection device 144 may be utilized to monitor many various characteristics relating to the motion of a user and/or device 100. Further, it will be appreciated that any abnormal activity or motion may be reported to a third party, including a family member (e.g., in the case of a fall), a safety monitoring service, or another agency.

The device 100 may be equipped with a location determining device 146 electrically coupled to the processing unit 128. The location determining device 146 may comprise instrumentality for determining the geographical position of the device 100. The location determining device 146 may comprise a Global Positioning System (GPS) device, such as a GPS receiver. A GPS receiver may be utilized to monitor the movement of a user. For example, the device 100 may be in first vicinity at a first time, and in second vicinity at a second time. By reporting the position of the device 100 to the processing unit 128, the device 100 may be able to monitor the movement of a user.

In one example, the user's movement may be examined to determine the distance the user has traveled from the first vicinity to the second vicinity while engaging in exercise, such as distance running. In this instance, the device 100 may report data of interest to the user, such as calories burned, or the like. In another instance, a user's lack of movement over time may be monitored. In this instance, an alert message may be delivered to the user (e.g., a wake up call) or to a third party (e.g., a health monitoring service) when movement of the user ceases (or is substantially limited) for a period of time.

The device 100 may comprise a sensing system for measuring a physiological condition/response through manipulation of an output of the device 100 and analysis of a user response. The sensing system may comprise medical sensors that are integral to the device 100. A user may request that the device 100 utilize the sensing system to perform a physiological measurement. Alternatively, the device 100 may perform a measurement surreptitiously. It will be appreciated that a number of requested and/or surreptitious measurements may be taken over time, and the results may be analyzed to determine patterns and signs of a user's status that would not otherwise be readily apparent. Further, measurements may be taken based upon a user's history. A variety of information gathering and statistical techniques may be utilized to optimize the gathering of such information and its subsequent analysis. It will be appreciated that the device 100 may utilize a variety of techniques to establish the identity of a user in relation to the gathering of such information. Once the identity of a user has been established, the device may record and monitor data appropriately for that user.

The device 100 may retain separate sets of information for a variety of users. Further, it is contemplated that the device 100 may correlate information about a particular user to information about other users in a related grouping (e.g., other user's having a familial relationship). This related information may be collected by the device 100 when it is utilized by more than one party. For example, a number of children in a family may share a telephone. If the telephone identifies one of the children as having a fever, it may report that information to the family, as well as monitoring and reporting that the other two children do not have a fever. It will be appreciated that such a report may comprise information regarding the timing of the measurements, and the expected accuracy (confidence interval) of the measurements. It is contemplated that time histories may be developed and viewed on the phone and/or transmitted off the device as needed.

It is contemplated that information about a user may be collected by another device. Further, data from another device may be transmitted to the device 100 and analyzed by the processing unit 128. External data may be analyzed in comparison with measurements taken by the device 100. External data may also be analyzed in view of a known or suspected user status as determined by the device 100. For example, information regarding a user's heart rate collected by another device may be uploaded to the device 100 and compared with information about the user's respiration collected by the device 100 and/or information inferred about the user's heart based on a physiological measurement collected by the device 100. Alternatively, the data from the device 100 may be uploaded to a central authority for comparison with data measured by other devices for the same user, for related users (e.g., family), or for entirely unrelated users, such as to establish health trends for a population, or the like.

The device 100 may be utilized to measure the hearing capability of a user. The speaker 122 may be utilized for providing various auditory cues to the user. Thus, the hearing capability of a user may be measured through manipulation of a volume of an audio output of the device 100. For example, in the case of the cellular telephone, the volume of the telephone's ring may be adjusted until the user responds to the ring volume. Alternatively, the hearing capability of a user may be measured through manipulation of a frequency of an audio output of the device 100. For example, in the case of the cellular telephone, the frequency of the telephone's ring may be adjusted until the user responds to the ring frequency. The manipulation of the ring volume and the ring frequency are explanatory only and not meant to be restrictive. It is contemplated that the output of the speaker 122 may be adjusted in a variety of ways, and various responses of a user may be interpreted in a variety of ways, in order to determine information about the user's status.

The device 100 may be utilized to measure the vision capability of a user. The display 120 may be utilized for providing various visual cues to the user. A font size of a text output of the device 100 may be manipulated to measure the vision capability of the user. For example, text may be provided at a first text size. If the user is capable of reading the first text size, the size may be adjusted to a second text size. The second text size may be smaller than the first text size. The text size may be adjusted until the user can no longer read the text with at least substantial accuracy. This information may be utilized to make a determination regarding the visual abilities of the user.

Alternatively, the processing unit 128 may be electrically coupled to a visual projection device 158. The visual projection device 158 may be configured for projecting an image (e.g., the text output of the device 100) onto a surface (e.g., a wall/screen). The vision capability of a user may be measured through manipulation of the image upon the surface. For example, text may be alternatively provided at a first text size and a second text size as previously described. It will be appreciated that the device 100 may measure the distance of the user away from the device 100 and/or the surface, (e.g., utilizing the camera). Alternatively, a user may inform the device of the distance. Further, the device 100 may provide a user with a desired distance and assume the user is at that distance. Any one of the aforementioned distance measurements/estimates may be factored into a determination of the vision capability of a user.

The text output of the device 100 may comprise labels for graphical buttons/icons provided on the display 120 (e.g., in an example where the display 120 comprises a touch screen). In one instance, the size of the text comprising the labels on a touch screen is adjusted to measure a user's vision by recording how accurate the user is at identifying the graphical buttons/icons. In another instance, the text output of the device 100 comprises an OLED label displayed on a button 114, and the text size of the button's label is adjusted through the OLED's output to measure the user's vision by recording how accurately button presses are made at various text sizes. In another example, the labels and/or on-screen placement for graphical buttons/icons may be altered in a pseudorandom fashion to prevent the user from memorizing the position of various labels/icons (e.g., in the case of testing visual recognition of various text sizes) and/or to test a user's mental acuity at identifying graphical buttons/icons at various and changing locations.

Alternatively, the text output of the device 100 may comprise labels for graphical buttons/icons projected by the visual projection device 158 upon a work surface (e.g., a desk at which a user may sit). The device 100 may utilize the camera or another device to record a user's motion proximal to a graphical button/icon projected by the visual projection device 158. The size of the text comprising the labels on the projected image may be adjusted to measure a user's vision by recording how accurate the user is at identifying the graphical buttons/icons, as previously described. Further, the locations of the graphical buttons/icons may be altered in a pseudorandom fashion as previously described.

Various data recorded about the user's recognition of the text output may be reported to the processing unit 128, and the processing unit 128 may make a determination about the user's vision utilizing a variety of considerations as required (e.g., the distance of the user from the device 100 as previously described). Further, it will be appreciated that other various symbols and indicia besides text may be utilized with the display 120 and/or the buttons 114 to measure the vision capability of a user, including placing lines of varying lengths, thicknesses, and/or angles on the display 120 as needed.

The device 100 may be utilized to measure the dexterity and/or reaction time of a user. The dexterity of a user may be measured through manipulation of the device 100 via a user input. For example, the processing unit 128 may be configured for measuring the dexterity of a user by examining characteristics of a depression of a button 114 (e.g., measurements of button press timing). In one instance, the device 100 provides the user with an output at time t₆, such as an audio cue provided by the speaker 122, a visual cue provided by the display 120, or another type of output as needed. The user may respond at a time t₇, providing a first reaction time Δ₁ between the cue and the response. Alternatively, the user may respond at time t₈, providing a second reaction time Δ₂ between the cue and the response. A reaction time of the user may be monitored to gather information about the status of the user. This information may be collected over time, or collected during a group of measurements during a period of time. An increase or decrease in a reaction time may be utilized to infer information about the user's status.

The device 100 may be utilized to measure characteristics of a user's memory. For example, a user's memory capability may be measured by the device 100. The device may store information known to a user at a certain point in time (e.g., information input or studied by the user). The information may then be stored in the memory 130 for subsequent retrieval. Upon retrieving the information, the processing unit 128 may provide questions/clues regarding the information to the user utilizing any of the devices that may be connected thereto. The user may then be prompted to supply the information to the device. By comparing user responses to the information stored in the memory 130, the device 100 may be able to make a determination regarding the memory capability of the user. This information may be collected over time, or collected during a group of measurements during a period of time. Further, the device 100 may be utilized to measure mental and/or physical characteristics by measuring how quickly tasks are completed on the device (e.g., typing a phone number) and/or external to the device (e.g., traveling from one location to another).

Measurements of a user's status may be taken according to a pseudorandom time scheme, or according to another technique for providing measurements at variously different time intervals. A first measurement may be taken at time to, a second measurement may be taken at time t₁, and a third measurement may be taken at time t₂. Times t₀, t₁, and t₂ may be separated by variously different time intervals according to a pseudorandom time scheme (e.g., a sequence of numbers that appears random but may have been generated by a finite computation). The processing unit 128 may measure the status of a user (e.g., a measurement of a physiological condition) through any one of the various components connected to it as described herein. The processing unit 128 may generate a sequence of pseudorandom numbers. Alternatively, the device 100 may receive a randomized seed or a sequence of pseudorandom numbers from an external source, which may utilize an environmental factor, or the like, to compute the random seed or the pseudorandom sequence.

Measurements of a user's status may be taken when available/opportunistically (i.e., when the device is held in a user's hand, when the device is open and aimed at a user's face, when the device is close to a user, when the device is close to a user's heart, when the device is gripped in a certain way). A fourth measurement may be taken at time t₃ and a fifth measurement may be taken at time t₄. The fourth and fifth measurements may comprise measuring a user's heart rate when the user is gripping the device 100. Times t₃ and t₄ may be separated by variously different time intervals according to a pseudorandom time scheme as previously described. However, times t₃ and t₄ are both within a measurement availability window. The measurement availability may be determined by the device 100 (e.g., measurements are taken when the device is in an “on” state as opposed to an “off” state). Alternatively, a user (either the user of the device 100 or another party) may determine the measurement availability. The processing unit 128 may measure the status of a user (e.g., a measurement of a physiological condition) through any one of the various components connected to it as described herein.

Alternatively, measurements of a user's status may be taken when requested. A sixth measurement may be taken at time t₅. Time t₅ may be subsequent to a measurement request. Time t₅ may be separated from the measurement request by variously different time intervals according to a pseudorandom time scheme as previously described. Alternatively, time t₅ may be determined by the device 100 (e.g., a measurement is taken when scheduled by the processing unit 128). It will be appreciated that a user (either a user of the device 100 or another party) may request the measurement. The processing unit 128 may measure the status of a user (e.g., a measurement of a physiological condition) through any one of the various components connected to it as described herein.

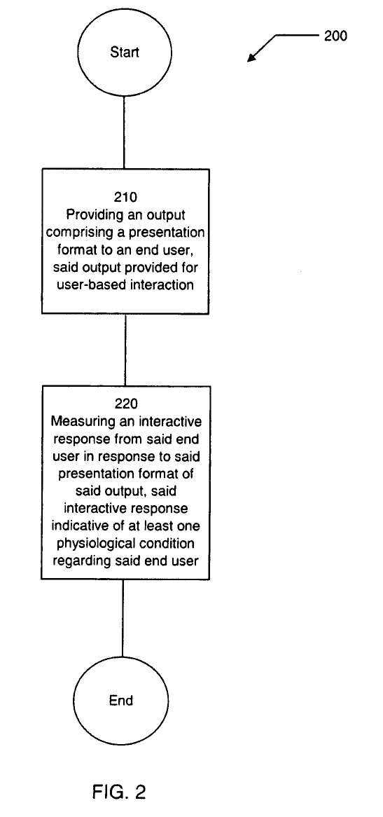

FIG. 2 illustrates an operational flow 200 representing example operations related to measuring at least one physiological condition for an end user. In FIG. 2 and in following figures that include various examples of operational flows, discussion and explanation may be provided with respect to the above-described examples of FIG. 1, and/or with respect to other examples and contexts. However, it should be understood that the operational flows may be executed in a number of other environments and contexts, and/or in modified versions of FIG. 1. Also, although the various operational flows are presented in the sequence(s) illustrated, it should be understood that the various operations may be performed in other orders than those which are illustrated, or may be performed concurrently.

After a start operation, the operational flow 200 moves to a providing operation 210, where an output including a presentation format may be provided to an end user, the output provided for user-based interaction. For example, as shown in FIG. 1, the device 100 may include a display 120 for providing a video output, a speaker 122 for providing an audio output, and/or a visual projection device 158 for providing a projected visual output. The output from the device 100 may have a presentation format that generally represents the form of information presented to the end user, including but not limited to characteristics such as appearance, arrangement, composition, layout, order, organization, orientation, pattern, proportion, shape, size, structure, style, and/or type. For example, the presentation format of the output from the device 100 may comprise a font size for text output by the display 120 and/or the visual projection device 158. Alternatively, the presentation format may comprise a volume and/or frequency for audio output by the speaker 122. It will be appreciated that the presentation formats disclosed herein are not meant to be exhaustive nor restrictive, and other outputs having varying presentation formats may be utilized as well, without departing from the scope and intent of the present disclosure.

Then, in a measuring operation 220, an interactive response from the end user may be measured in response to the presentation format of the output, the interactive response indicative of at least one physiological condition regarding the end user. For example, as shown in FIG. 1, the end user may interact with the device by pressing a button 114. Alternatively, the end user may issue an audible command or a command sequence to a microphone 116. The interactive response from the end user may be indicative of at least one physiological condition regarding the end user. For instance, one interactive response may be indicative of a hearing condition regarding the end user (e.g., an interactive response directing the device 100 to increase the volume of an audio output by the speaker 122), while another interactive response may be indicative of a vision condition regarding the end user (e.g., an interactive response directing the device 100 to increase the font size for text output by the display 120).

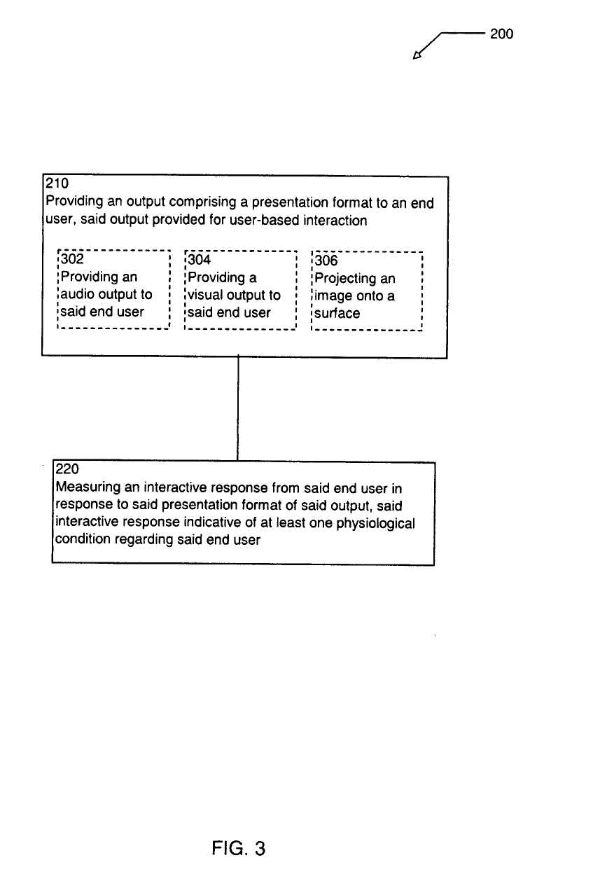

FIG. 3 illustrates alternative embodiments of the example operational flow 200 of FIG. 2. FIG. 3 illustrates example embodiments where the providing operation 210 may include at least one additional operation. Additional operations may include an operation 302, an operation 304, and/or an operation 306.

At the operation 302, an audio output may be provided to the end user. For example, as shown in FIG. 1, the speaker 122 may be utilized to provide the end user with an audio output comprising, for instance, music, voice data, tones (e.g., from a tone generator), and/or other auditory information as needed.

At the operation 304, a visual output may be provided to the end user. For example, as shown in FIG. 1, the display 120 may be utilized to provide the end user with a visual output comprising, for instance, text, graphics, symbols, indicia, and/or other visual information as needed.

At the operation 306, an image may be projected onto a surface. For example, as shown in FIG. 1, the visual projection device 158 may be utilized to provide the end user with a projected visual output comprising, for instance, text, graphics, symbols, indicia, and other visual information as needed.

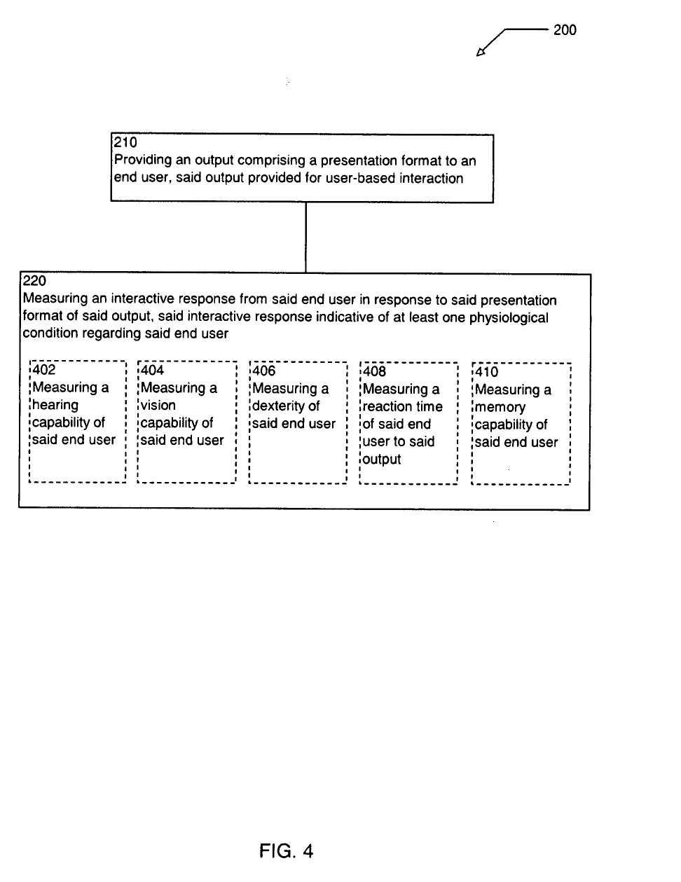

FIG. 4 illustrates alternative embodiments of the example operational flow 200 of FIG. 2. FIG. 4 illustrates example embodiments where the measuring operation 220 may include at least one additional operation. Additional operations may include an operation 402, an operation 404, an operation 406, an operation 408, and/or an operation 410.

At the operation 402, a hearing capability of the end user may be measured. For example, as shown in FIG. 1, the end user may respond to audio provided via the speaker 122. The end user's interactive response may be indicative of at least one physiological condition. For instance, one interactive response may be indicative of hearing loss, while another interactive response may be indicative of increased auditory sensitivity.

At the operation 404, a vision capability of the end user may be measured. For example, as shown in FIG. 1, the end user may respond to visuals provided via the display 120. The end user's interactive response may be indicative of at least one physiological condition. For instance, one interactive response may be indicative of vision loss, while another interactive response may be indicative of visual acuity (e.g., nearsightedness and/or farsightedness).

At the operation 406, dexterity of the end user may be measured. For example, as shown in FIG. 1, the end user may provide an interactive response via the buttons 114 provided with the keyboard 122. The end user's interactive response may be indicative of at least one physiological condition. For instance, an interactive response may be indicative of manual dexterity (e.g., typing speed and/or accuracy).

At the operation 408, a reaction time of the end user to the output may be measured. For example, as shown in FIG. 1, the end user may respond to an output from one or more of the speaker 122, the display 120, and/or the visual projection device 158. By measuring how quickly the end user responds to the output, a reaction time for the end user may be determined (e.g., by the processing unit 128).

At the operation 410, a memory capability of the end user may be measured. For example, as shown in FIG. 1, one or more of the speaker 122, the display 120, and/or the visual projection device 158 may be utilized to provide information to the end user. By measuring how accurately the end user recalls the information, a memory capability for the end user may be determined (e.g., by the processing unit 128).

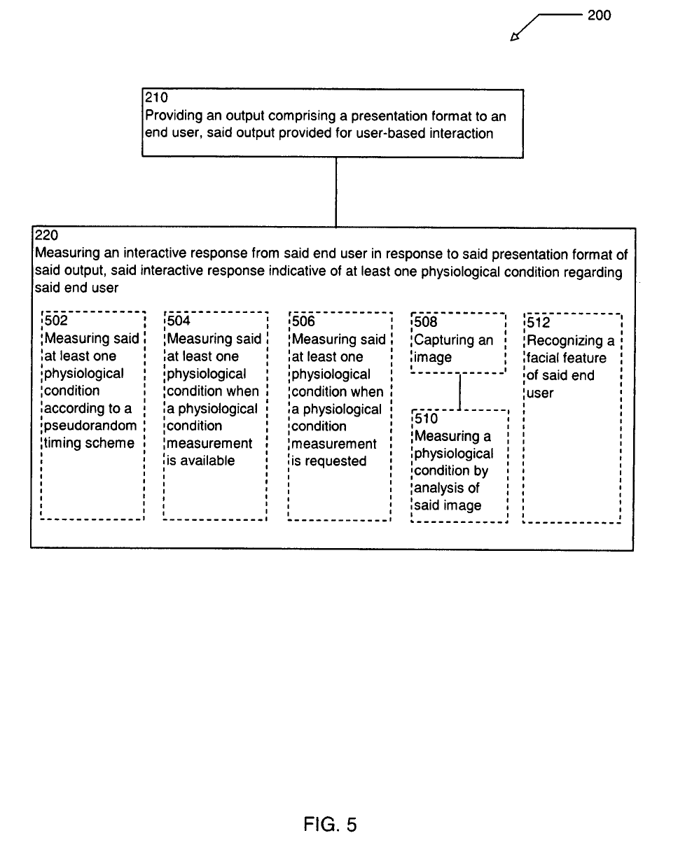

FIG. 5 illustrates alternative embodiments of the example operational flow 200 of FIG. 2. FIG. 5 illustrates example embodiments where the measuring operation 220 may include at least one additional operation. Additional operations may include an operation 502, an operation 504, an operation 506, an operation 508, an operation 510, and/or an operation 512.

At the operation 502, at least one physiological condition may be measured according to a pseudorandom timing scheme. For example, as shown in FIG. 1, the processing unit 128 may be utilized to generate pseudorandom timing information, and a measurement of information related to the physiology of the end user may be taken according to the pseudorandom timing information.

At the operation 504, at least one physiological condition may be measured when a physiological condition measurement is available. For example, as shown in FIG. 1, a measurement of information related to the physiology of the end user may be taken after a determination of the availability of the measurement is made. For instance, a measurement may be taken when the device 100 is in an on state.

At the operation 506, at least one physiological condition may be measured when a physiological condition measurement is requested. For example, as shown in FIG. 1, the end user may request a measurement utilizing the buttons 114 of the keyboard 112 (or another interface), and the device 100 may then proceed to take a measurement.

At the operation 508, an image may be captured. For example, as shown in FIG. 1, the image capture device 132 (e.g., a camera) may be utilized to capture an image of the end user's face. Then, at the operation 510, a physiological condition may be measured by analyzing the image. For example, as shown in FIG. 1, the processing unit 128 may be utilized to analyze facial features of the image and make a determination regarding the end user. In one specific embodiment, a determination is made regarding the health and/or wellbeing of the end user by analyzing his or her complexion.

At the operation 512, a facial feature of the end user may be recognized. For example, as shown in FIG. 1, the image capture device 132 (e.g., a camera) may be utilized to capture an image of the end user's face. Then, the processing unit 128 may be utilized to analyze facial features of the image and make a determination regarding the identity of the end user.

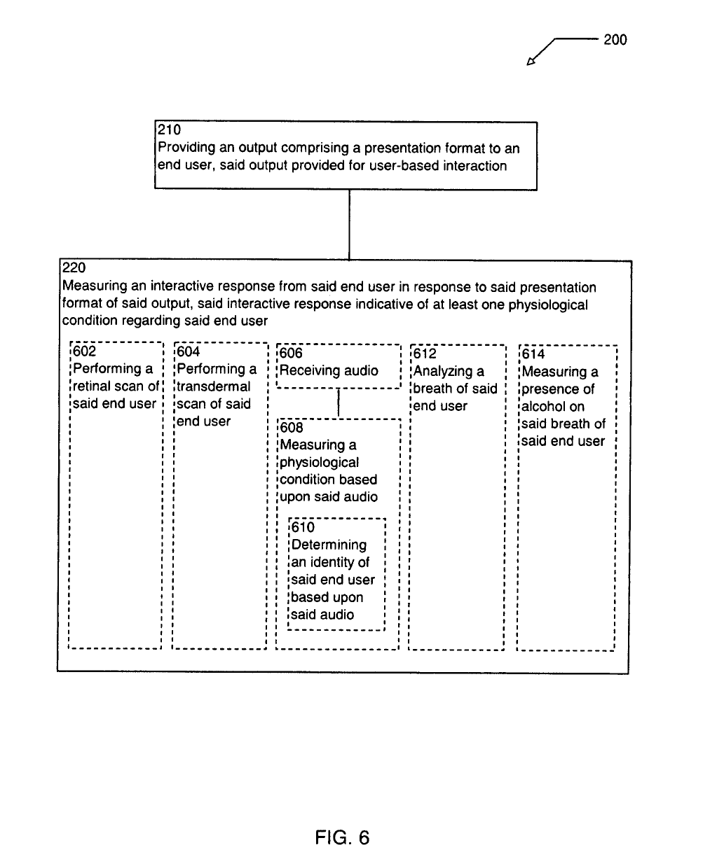

FIG. 6 illustrates alternative embodiments of the example operational flow 200 of FIG. 2. FIG. 6 illustrates example embodiments where the measuring operation 220 may include at least one additional operation. Additional operations may include an operation 602, an operation 604, an operation 606, an operation 608, an operation 610, an operation 612, and/or an operation 614.

At the operation 602, a retinal scan of the end user may be performed. For example, as shown in FIG. 1, the image capture device 132 (e.g., a camera) may be utilized to capture an image of the end user's retina. Then, the processing unit 128 may be utilized to analyze retinal features of the image and make a determination regarding the identity of the end user.

At the operation 604, a transdermal scan of the end user may be performed. For example, as shown in FIG. 1, the image capture device 132 (e.g., a camera) may be utilized to capture an image of or through the end user's skin. Then, the processing unit 128 may be utilized to analyze the image and make a determination regarding the health and/or wellbeing of the end user (e.g., blood sugar) by measuring aspects of the image.

At the operation 606, audio may be received. For example, as shown in FIG. 1, the microphone 116 may be utilized to receive an interactive response comprising voice information from the end user. Then, at the operation 608, a physiological condition may be measured based upon the audio. For example, as shown in FIG. 1, the processing unit 128 may be utilized to analyze the voice information and determine information regarding the health and/or wellbeing of the user (e.g., by calculating a voice-stress level or the like). Further, at the operation 610, an identity of the end user may be determined based upon the audio. For example, as shown in FIG. 1, the audio received by the microphone 116 may be examined to identify vocal characteristics unique to the end user.

At the operation 612, a breath of the end user may be analyzed. For example, as shown in FIG. 1, the breath analyzer 142 may be utilized to receive a breath from the end user. The processing unit 128 may be utilized to analyze the breath and make a determination about the end user's health and/or well being (e.g., a blood-alcohol level). For example, at the operation 614, a presence of alcohol on the breath of the end user may be measured.

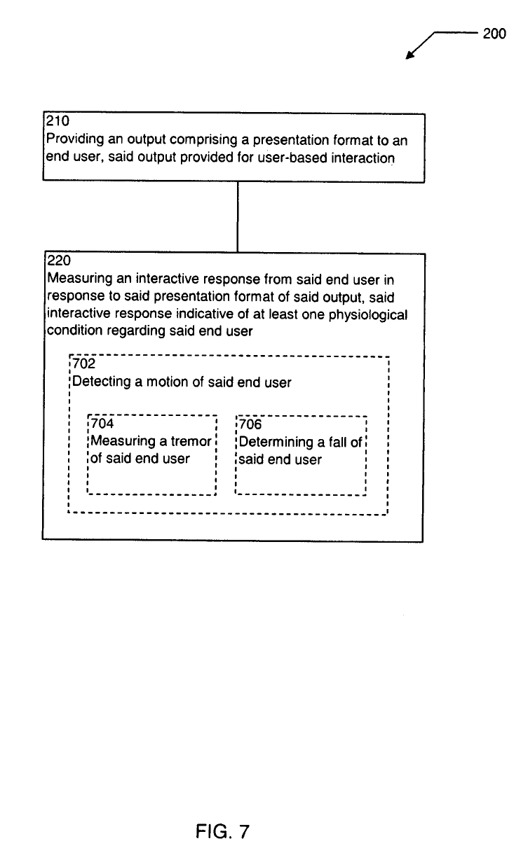

FIG. 7 illustrates alternative embodiments of the example operational flow 200 of FIG. 2. FIG. 7 illustrates example embodiments where the measuring operation 220 may include at least one additional operation. Additional operations may include an operation 702, an operation 704, and/or an operation 706.

At the operation 702, a motion of the end user may be detected. For example, as shown in FIG. 1, the motion detection device 144 may be utilized to measure motion of the end user. Further, at the operation 704, a tremor of the end user may be measured. For example, as shown in FIG. 1, the motion detection device 144 may measure motions characterizing a tremor when the device 100 is held and/or worn by the end user. Alternatively, at the operation 706, a fall of the end user may be determined. For example, as shown in FIG. 1, the motion detection device 144 may measure a rapid acceleration followed by a rapid deceleration, which may be indicative of a fall.

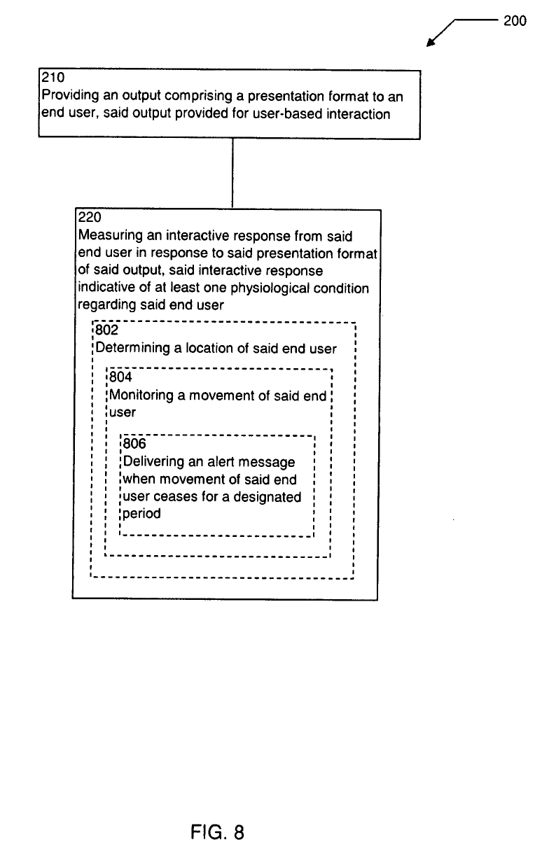

FIG. 8 illustrates alternative embodiments of the example operational flow 200 of FIG. 2. FIG. 8 illustrates example embodiments where the measuring operation 220 may include at least one additional operation. Additional operations may include an operation 802, an operation 804, and/or an operation 806.

At the operation 802, a location of the end user may be determined. For example, as shown in FIG. 1, the location determining device 146 may be utilized to determine a geographic position for the end user. Further, at the operation 804, a movement of the end user may be monitored. For example, as shown in FIG. 1, the location determining device 146 may periodically report positions for the end user to the processing unit 128, which may monitor movement of the end user over time. Moreover, at the operation 806, an alert message may be delivered when movement of the end user ceases for a designated period. For example, as shown in FIG. 1, the location determining device 146 may periodically report positions for the end user to the processing unit 128, which may identify when movement of the end user has ceased for a designated period.

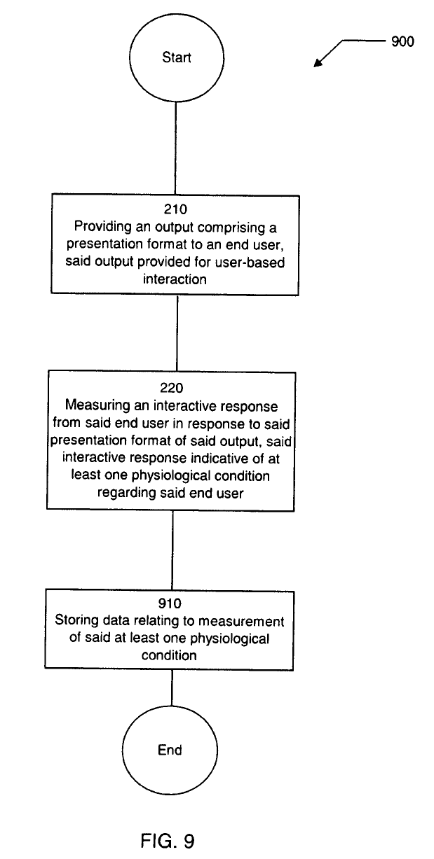

FIG. 9 illustrates an operational flow 900 representing example operations related to measuring at least one physiological condition for an end user. FIG. 9 illustrates an example embodiment where the example operational flow 200 of FIG. 2 may include at least one additional operation 910. After a start operation, a providing operation 210, and a measuring operation 220, the operational flow 900 moves to a storing operation 910, where data relating to measurement of the at least one physiological condition may be stored. For example, as shown in FIG. 1, the memory 128 may store information regarding a physiological condition of the end user.

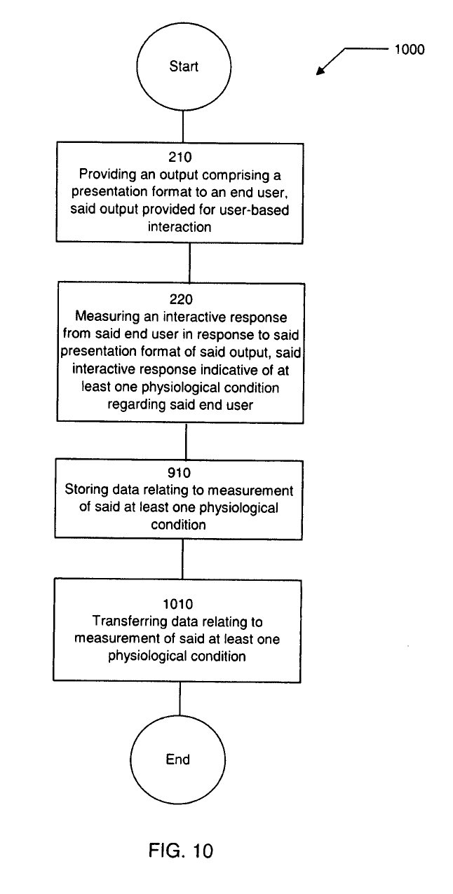

FIG. 10 illustrates an operational flow 1000 representing example operations related to measuring at least one physiological condition for an end user. FIG. 10 illustrates an example embodiment where the example operational flow 200 of FIG. 2 may include at least one additional operation 1010. After a start operation, a providing operation 210, and a measuring operation 220, the operational flow 1000 moves to a transferring operation 1010, where data relating to measurement of the at least one physiological condition may be transferred. For example, as shown in FIG. 1, the data transfer interface 124 may transfer information regarding a physiological condition of the end user.

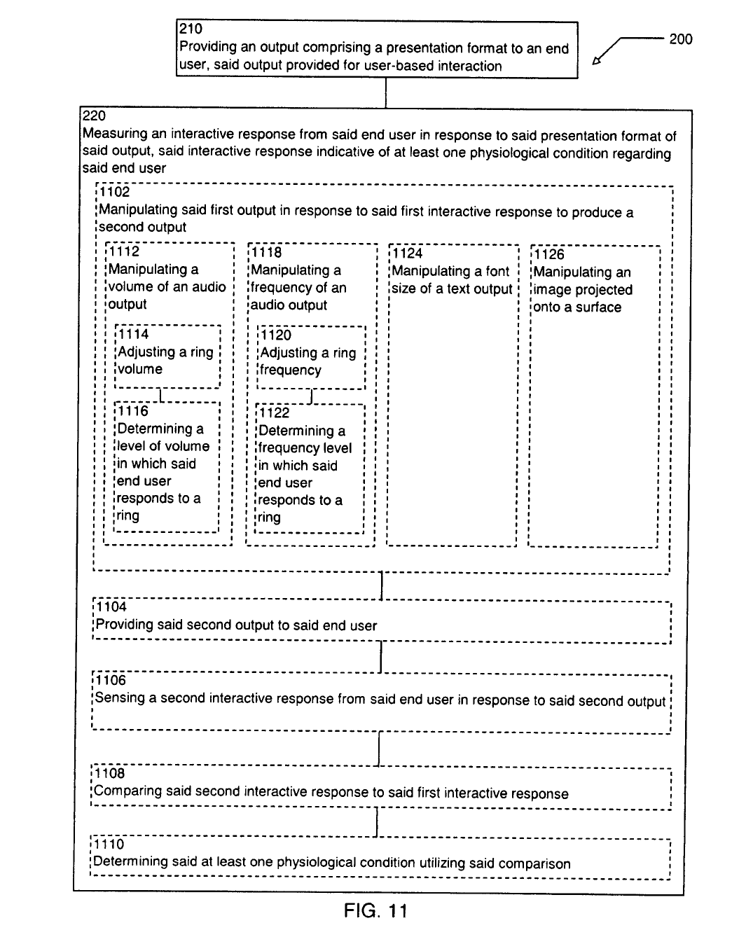

FIG. 11 illustrates alternative embodiments of the example operational flow 200 of FIG. 2. FIG. 11 illustrates example embodiments where the measuring operation 220 may include at least one additional operation. Additional operations may include an operation 1102, an operation 1104, an operation 1106, an operation 1108, an operation 1110, an operation 1112, an operation 1114, an operation 1116, an operation 1118, an operation 1120, an operation 1122, an operation 1124, and/or an operation 1126.

At the operation 1102 a first output may be manipulated in response to a first interactive response to produce a second output. For example, as shown in FIG. 1, the speaker 122 may provide the end user with an audio output. The end user may provide a first interactive response comprising a request to increase the volume of the audio provided by the speaker 122. Based on the first interactive response, the processing unit 128 may direct the device 100 to increase the volume of the speaker 122 by an incremental level, providing a second output comprising another audio output having an increased volume level. Then, at the operation 1104, the second output may be provided to the end user. For example, as shown in FIG. 1, the speaker 122 may provide the second output to the end user at the increased volume level. Next, at the operation 1106, a second interactive response may be sensed from the end user in response to the second output. For example, as shown in FIG. 1, the end user may utilize a button 114 provided with the keyboard 112 (or another interface) to provide a second interactive response comprising another desired increase in volume. Then, at the operation 1108, the second interactive response may be compared to the first interactive response. For example, as shown in FIG. 1, the processing unit 128 may compare the first interactive response to the second interactive response. Next, at the operation 1110, the at least one physiological condition may be determined utilizing a comparison. For example, as shown in FIG. 1, the comparison of the first interactive response to the second interactive response by the processing unit 128 may allow the device 100 to make a determination about the hearing capability of the end user (e.g., the end user may suffer from a hearing loss).

Further, at the operation 1112, a volume of an audio output may be manipulated. For example, as shown in FIG. 1, the processing unit 128 may direct the device 100 to increase the volume level provided by the speaker 122. Moreover, at the operation 1114, a ring volume may be adjusted. For example, as shown in FIG. 1, the device 100 may increase the volume level of a ring provided by the speaker 122 (e.g., in a case where the device 100 comprises a mobile telephone). Then, at the operation 1116, a level of volume in which the end user responds to a ring may be determined. For example, as shown in FIG. 1, the processing unit 128 may increase the volume of a ring provided by the speaker 122 until an interactive response by the end user comprises a response to the ring.

Alternatively, at the operation 1118, a frequency of an audio output may be manipulated. For example, as shown in FIG. 1, the processing unit 128 may direct the device 100 to increase the frequency level provided by the speaker 122. Moreover, at the operation 1120, a ring frequency may be adjusted. For example, as shown in FIG. 1, the device 100 may increase the frequency level of a ring provided by the speaker 122 (e.g., in a case where the device 100 comprises a mobile telephone). Then, at the operation 1122, a frequency level in which the end user responds to a ring may be determined. For example, as shown in FIG. 1, the processing unit 128 may increase the frequency of a ring provided by the speaker 122 until an interactive response by the end user comprises a response to the ring.

Further, at the operation 1124, a font size of a text output may be manipulated. For example, as shown in FIG. 1, the display 120 and/or the visual projection device 158 may adjust the font size of a text output to determine a user's visual acuity (e.g., farsightedness and/or nearsightedness).

Alternatively, at the operation 1126, an image projected onto a surface may be manipulated. For example, as shown in FIG. 1, the visual projection device 158 may adjust the font size of a projected text output to determine a user's visual acuity (e.g., farsightedness and/or nearsightedness).

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk'(DVD), a digital tape, a computer memory, etc.; and a transmission type medium such as a digital and/or an analog communication medium (e.g., a fiber optic cable, a waveguide, a wired communications link, a wireless communication link, etc.).

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

Those skilled in the art will recognize that it is common within the art to describe devices and/or processes in the fashion set forth herein, and thereafter use engineering practices to integrate such described devices and/or processes into image processing systems. That is, at least a portion of the devices and/or processes described herein can be integrated into an image processing system via a reasonable amount of experimentation. Those having skill in the art will recognize that a typical image processing system generally includes one or more of a system unit housing, a video display device, a memory such as volatile and non-volatile memory, processors such as microprocessors and digital signal processors, computational entities such as operating systems, drivers, and applications programs, one or more interaction devices, such as a touch pad or screen, control systems including feedback loops and control motors (e.g., feedback for sensing lens position and/or velocity; control motors for moving/distorting lenses to give desired focuses. A typical image processing system may be implemented utilizing any suitable commercially available components, such as those typically found in digital still systems and/or digital motion systems.

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

1. A method comprising:
providing an output including a presentation format to an end user, said output provided for user-based interaction; and
measuring an interactive response from said end user in response to said presentation format of said output, said interactive response indicative of at least one physiological condition regarding said end user.

2. The method of claim 1, wherein said providing an output including a presentation format to an end user comprises:
providing an audio output to said end user.

3. The method of claim 1, wherein said measuring an interactive response from said end user in response to said presentation format of said output comprises:
measuring a hearing capability of said end user.

4. The method of claim 1, wherein said providing an output including a presentation format to an end user comprises:
providing a visual output to said end user.

5. The method of claim 1, wherein said measuring an interactive response from said end user in response to said presentation format of said output comprises:
measuring a vision capability of said end user.

6. The method of claim 1, wherein said measuring an interactive response from said end user in response to said presentation format of said output comprises:
measuring a dexterity of said end user.

7. The method of claim 1, further comprising:
storing data relating to measurement of said at least one physiological condition.

8. The method of claim 1, further comprising:
transferring data relating to measurement of said at least one physiological condition.

9. The method of claim 1, wherein said measuring an interactive response from said end user in response to said presentation format of said output comprises:
measuring a reaction time of said end user to said output.

10. The method of claim 1, wherein said measuring an interactive response from said end user in response to said presentation format of said output comprises:
measuring a memory capability of said end user.

11. The method of claim 1, wherein said measuring an interactive response from said end user in response to said presentation format of said output comprises:
measuring said at least one physiological condition according to a pseudorandom timing scheme.

12. The method of claim 1, wherein said measuring an interactive response from said end user in response to said presentation format of said output comprises:
measuring said at least one physiological condition when a physiological condition measurement is available.

13. The method of claim 1, wherein said measuring an interactive response from said end user in response to said presentation format of said output comprises:
measuring said at least one physiological condition when a physiological condition measurement is requested.

14. The method of claim 1, wherein said measuring an interactive response from said end user in response to said presentation format of said output comprises:
capturing an image; and
measuring a physiological condition by analysis of said image.

15. The method of claim 1, wherein said measuring an interactive response from said end user in response to said presentation format of said output comprises:
recognizing a facial feature of said end user.

16. The method of claim 1, wherein said measuring an interactive response from said end user in response to said presentation format of said output comprises:
performing a retinal scan of said end user.

17. The method of claim 1, wherein said measuring an interactive response from said end user in response to said presentation format of said output comprises:
performing a transdermal scan of said end user.

18. The method of claim 1, wherein said measuring an interactive response from said end user in response to said presentation format of said output comprises:
receiving audio; and
measuring a physiological condition based upon said audio.

19. The method of claim 18, wherein said measuring a physiological condition based upon said audio comprises:
determining an identity of said end user based upon said audio.

20. The method of claim 1, wherein said measuring an interactive response from said end user in response to said presentation format of said output comprises:
analyzing a breath of said end user.

21. The method of claim 20, wherein said analyzing a breath of said end user comprises:
measuring a presence of alcohol on said breath of said end user.

22. The method of claim 1, wherein said measuring an interactive response from said end user in response to said presentation format of said output comprises:
detecting a motion of said end user.

23. The method of claim 22, wherein said detecting a motion of said end user comprises:
measuring a tremor of said end user.

24. The method of claim 22, wherein said detecting a motion of said end user comprises:
determining a fall of said end user.

25. The method of claim 1, wherein said measuring an interactive response from said end user in response to said presentation format of said output comprises:
determining a location of said end user.

26. The method of claim 25, wherein said determining a location of said end user comprises:
monitoring a movement of said end user.

27. The method of claim 26, wherein said monitoring a movement of said end user comprises:
delivering an alert message when movement of said end user ceases for a designated period.

28. The method of claim 1, wherein said providing an output including a presentation format to an end user comprises:
projecting an image onto a surface.

29. The method of claim 1, wherein said measuring an interactive response from said end user in response to said presentation format of said output comprises:
manipulating a first output in response to a first interactive response to produce a second output;
providing said second output to said end user;
sensing a second interactive response from said end user in response to said second output;
comparing said second interactive response to said first interactive response; and
determining said at least one physiological condition utilizing a comparison.

30. The method of claim 29, wherein said manipulating a first output in response to a first interactive response to produce a second output comprises:
manipulating a volume of an audio output.

31. The method of claim 30, wherein said manipulating a volume of an audio output comprises:
adjusting a ring volume; and
determining a level of volume in which said end user responds to a ring.

32. The method of claim 29, wherein said manipulating a first output in response to a first interactive response to produce a second output comprises:
manipulating a frequency of an audio output.

33. The method of claim 32, wherein said manipulating a frequency of an audio output comprises:
adjusting a ring frequency; and
determining a frequency level in which said end user responds to a ring.

34. The method of claim 29, wherein said manipulating a first output in response to a first interactive response to produce a second output comprises:
manipulating a font size of a text output.

35. The method of claim 29, wherein said manipulating a first output in response to a first interactive response to produce a second output comprises:
manipulating an image projected onto a surface.

36. A system comprising:
means for providing an output including a presentation format to an end user, said output provided for user-based interaction; and
means for measuring an interactive response from said end user in response to said presentation format of said output, said interactive response indicative of at least one physiological condition regarding said end user.

37-70. (canceled)

71. A system comprising:
circuitry for providing an output including a presentation format to an end user, said output provided for user-based interaction; and
circuitry for measuring an interactive response from said end user in response to said presentation format of said output, said interactive response indicative of at least one physiological condition regarding said end user.
